(19) United States
(12) Patent Application Publication
(10) Pub. No.: US 2007/0250035 A1
(43) Pub. Date: Oct. 25, 2007

(54) Devices and methods for intravascular drug delivery

(76) Inventors: Fozan O. El-Nounou, Santa Clara, CA (US); Paul M. Consigny, San Jose, CA (US); Florian N. Ludwig, Mountain View, CA (US)

Correspondence Address: BLAKELY SOKOLOFF TAYLOR & ZAFMAN, 1279 OAKMEAD PARKWAY, SUNNYVALE, CA, 94085-4040, US

(21) Appl. No.: 11/407,707

(22) Filed: Apr. 19, 2006

(51) Int. Cl. A61M 31/00

(52) U.S. Cl. 604/509; 604/103.02; 604/173

(57) ABSTRACT

A catheter configured for delivering an agent to a patient's vessel wall, having self-expanding frame, and a method of delivering an agent to a patient's vessel wall. A first catheter has an elongated shaft with an agent delivery lumen and self-expanding frame on a distal shaft section formed of plurality of hollow tubes having joined first ends and free second ends, the free end of each hollow tube having an agent delivery port in fluid communication with the shaft agent delivery lumen and having a hooked tip configured for penetrating the vessel wall in the expanded configuration. Another catheter has a frame around the outer surface of a lining member such as a balloon or a tubular sleeve, such that the frame expanded against the vessel wall and the lining member together define a plurality of pockets between the vessel wall and the outer surface of the lining member.

### FIELD

The present invention relates generally to medical devices, and more particularly to a catheter for delivery of an agent to the coronary or peripheral vasculature.

### BACKGROUND OF THE INVENTION

In the treatment of diseased vasculature, therapeutic agents have commonly been administered, typically as part of other interventional therapies such as angioplasty or stent delivery. Local, as opposed to systemic delivery is a preferred method of treatment in that smaller total levels of medication are administered in comparison to systemic dosages, yet are concentrated at a specific site. As a result, local delivery produces fewer side effects and achieves more effective results.

A variety of methods and devices have been proposed for percutaneous drug delivery to a diseased region of the vasculature. For example, catheters having porous balloons can be used to deliver a therapeutic agent infused into the inflatable interior of the porous balloon and through the porous wall of the balloon. Alternatively, prostheses such as stents or other implantable devices provide for local drug delivery when coated or otherwise made to include a therapeutic agent which elutes from the implanted prosthesis. Another suggested method involves the use of one or more catheters having multiple balloons. The diseased region is isolated by inflating the balloons on either side of the diseased region, and the therapeutic agent is infused through a lumen of the catheter shaft and into the isolated diseased region from a delivery port on the catheter shaft located between the balloons.

One difficulty has been maximizing the amount of drug taken-up and retained at the diseased site, while minimizing the wash-out of large amounts of drug downstream of the treatment site. Drug wash-out reduces the efficiency of local intravascular drug delivery, in addition to causing potentially harmful systemic exposure to the drug. Therefore, it would be a significant advance to provide an improved device and method for providing therapy to a desired location within a patient's body lumen.

### SUMMARY OF THE INVENTION

The invention is directed to a catheter configured for delivering an agent to a patient's vessel wall, having self-expanding frame.

In a first embodiment, the catheter comprises an elongated shaft having an inner tubular member with at least one agent delivery lumen, and an outer sheath member slidably disposed on the inner member, and a self-expanding frame on a distal shaft section fixedly secured to the inner member and slidably disposed in the outer member in a radially collapsed configuration. The self-expanding frame radially expands to an expanded configuration by release of a radially restraining force of the outer member. The frame is formed of plurality of hollow tubes having joined first ends and free second ends, the free end of each hollow tube having an agent delivery port in fluid communication with the shaft agent delivery lumen and having a hooked tip configured for penetrating the vessel wall in the expanded configuration, to imbed the agent delivery port within the vessel wall or the periadventitia space outside the vessel wall in the expanded configuration. As a result, the catheter provides for direct injection of the agent to the vessel wall (or other target tissue) to minimize drug wash-out in the vasculature.

The frame comprises one or more circumferentially spaced, longitudinally extending hollow tubes, forming an open-walled, discontinuous structure of the frame. The deployed frame is thus configured to prevent or minimize interruption of blood flow in the main and any side branches of the patient's vessel during agent delivery along an extended length of the vessel. The open distal end of the frame (formed by the free ends of the hollow tubes) radially expanded into contact with the vessel wall provides for minimal disruption of fluid flow within the patient's body lumen.

In contrast to a microporous drug delivery balloon, the catheter operative distal section contacts the vessel wall only with the relatively thin hollow tubes of the self-expanding frame. As a result, the catheter preferably minimizes endothelial injury and prevents complete denudation of the delivery area within the vessel. Additionally, the tubes spaced apart around the circumference of the frame, unlike a drug delivery balloon, allow for the expanded frame to push into the vessel wall, to be at least partially enveloped by the wall in one embodiment.

In another embodiment, a catheter of the invention generally comprises an elongated shaft having an inner tubular member with an inflation lumen and an outer sheath member slidably disposed on the inner member, a balloon on a distal shaft section fixedly secured to the inner member such that the balloon has an interior in fluid communication with the inflation lumen for inflating the balloon to an inflated configuration, and a self-expanding frame on the distal shaft section fixedly secured to the inner member and slidably disposed in the outer member in a radially collapsed configuration which radially expands to an expanded configuration by release of a radially restraining force of the outer member. The frame is around the outer surface of the balloon such that the frame expanded against the vessel wall and the inflated balloon together define a plurality of pockets between the vessel wall and the outer surface of the inflated balloon. A plurality of agent delivery ports are along a distal portion of the catheter. As a result, the catheter lengthens agent retention time at the vessel wall, enhances the efficiency of agent uptake, and prevents or inhibits wash-out of the agent delivered through the ports and into the pockets defined by the expanded frame and the inflated balloon, preferably by at least partially containing the agent in the pockets. Compared to prior drug delivery systems, the surface area of the treated vessel is relatively large due to the pockets. In an alternative embodiment, the frame has a tubular sleeve fixedly secured to the frame, instead of the balloon, to function as a lining member for forming the pockets. The sleeve expands and collapses together with the frame, and thus avoids the need for delivering inflation fluid through the shaft. Therefore, although discussed below primarily in terms of the embodiment in which the lining member is a balloon, it should be understood that other lining member configurations can be used including the embodiment in which the lining member is a tubular sleeve.

The outer surface of the balloon (or other lining member) is separated from the vessel wall by the self-expanding frame therearound. Therefore, similar to the embodiment discussed above having a self-expanding frame of hollow tubes, the catheter operative distal section contacts the vessel wall only with, or primarily with, the relatively thin members of the self-expanding frame. Thus, injury to the vessel wall is minimized. Additionally, the expanded frame pushes into the wall to be at least partially enveloped by the wall in a presently preferred embodiment, to preferably optimize agent delivery. The embedded frame creates channels along the tissue wall which increase the surface contact area between the drug delivery distal section of the catheter and the tissue, and which function as reservoirs to lengthen the drug retention time on the lumen surface.

As discussed above, the self-expanding frame is configured to contact the vessel wall with the relatively thin hollow tubes or solid members of the frame, and thus minimizes injury to the vessel wall. Consequently, in one embodiment of a method of the invention, a self-expanding frame is slidably displaced in the expanded configuration within the body lumen to directly deliver agent to a longer length of the vessel. Unlike a porous balloon, or other drug delivery system which has a relatively large contact surface area extending around the circumference of the operative distal end of the device, the potential damage caused by moving the thin members of the frame is limited to only a small percentage of the inner circumference of the vessel wall. The method generally comprises advancing within the patient's vessel a catheter which has an elongated shaft having an inner tubular member and an outer sheath member slidably disposed on the inner member, and a self-expanding frame on the distal shaft section fixedly secured to the inner member, the frame being in a radially collapsed configuration within the outer member. The method includes radially expanding the frame into contact with a first section of the vessel wall by slidably displacing the frame relative to the outer member, so that the frame expands to an expanded configuration by release of a radially restraining force of the outer member. Agent is then delivered through a plurality of agent delivery ports along a distal portion of the catheter to deliver agent to the first section of the vessel wall. The method includes slidably displacing the frame in the expanded configuration longitudinally along the vessel to position the expanded frame at a second section of the vessel wall, and flowing agent through the agent delivery ports to deliver agent to the second section of the vessel wall.

Due to the self-expanding frame, a catheter of the invention has a relatively low profile and high flexibility, which facilitates positioning the operative distal end of the catheter within the vasculature. Although the primary target of the catheter is the proximal two thirds of the diseased coronaries in one embodiment, the catheter can be configured to allow for accessing the tortuous, narrow distal vasculature. In a presently preferred embodiment, a catheter of the invention is configured for delivery of an agent to a coronary artery or vein. However, the vasculature need not be coronary, and can be, for example, renal, femoral, popliteal, carotid, cerebral or other arteries and veins, aneurysms and aneurismal sacs, and may include delivery to implanted devices therein such as grafts, stents and the like. Similarly, agent delivery may occur to the wall of a variety of tubular body lumens including pulmonary, gastrointestinal and urinary tract structures. Thus, the term “vessel” as used herein should be understood to refer generally to body lumens.

A variety of suitable agents can be delivered using the catheter(s) and method(s) of the invention, including therapeutic and diagnostic agents. The agents are typically intended for treatment and/or diagnosis of coronary, neurovascular, and/or other vascular disease, and may be useful as a primary treatment of the diseased vessel, or alternatively, as a secondary treatment in conjunction with other interventional therapies such as angioplasty or stent delivery. Suitable therapeutic agents include, but are not limited to, thrombolytic drugs, anti-inflammatory drugs, anti-proliferative drugs, drugs restoring and/or preserving endothelial function, and the like. A variety of bioactive agents can be used including but not limited to peptides, proteins, oligonucleotides, cells, and the like. A variety of diagnostic agents can be used according to the present invention. According to the present invention, agents described herein may be provided in a variety of suitable formulations and carriers including liposomes, polymerosomes, nanoparticles, microparticles, lipid/polymer micelles, and complexes of agents with lipid and/or polymers, and the like.

A catheter of the invention provides for improved delivery of drug therapy to the patient's vessel wall, by enhancing drug uptake into the vessel wall while minimizing drug wash-out into the vascular system. These and other advantages of the invention will become more apparent from the following detailed description of the invention and accompanying exemplary drawings.

### BRIEF DESCRIPTION OF THE DRAWINGS

FIG. 1 is an elevational view, partially in section, of a catheter embodying features of the invention, having a self-expanding frame of hollow tubes with hooked ends, illustrating the frame in a collapsed configuration in a patient's body vessel.

FIGS. 2-4 are a transverse cross sectional views of the catheter of FIG. 1, taken along lines 2-2, 3-3, and 4-4, respectively.

FIG. 5 is an enlarged view of a hooked tip of the frame of FIG. 1.

FIG. 6 illustrates the catheter of FIG. 1 with the frame in an expanded configuration within the vessel.

FIG. 7 is an elevational view, partially in section, of an alternative catheter embodying features of the invention, having a self-expanding frame surrounding a balloon, illustrating the catheter in a collapsed configuration in a patient's body vessel.

FIGS. 8-10 are transverse cross sectional views of the catheter of FIG. 7, taken along lines 8-8, 9-9, and 10-10, respectively.

FIG. 11 illustrates the catheter of FIG. 7 with the balloon and frame in an expanded configuration in the vessel.

FIG. 12 is a transverse cross sectional view of the catheter of FIG. 11, taken along line 12-12.

FIG. 13 is a transverse cross section of an alternative embodiment, having a sleeve secured to an inner surface of the catheter frame

FIG. 14 is an enlarged view of one embodiment of an agent delivery port of the catheter frame, having a penetrating flap opening.

### DETAILED DESCRIPTION OF THE PREFERRED EMBODIMENTS

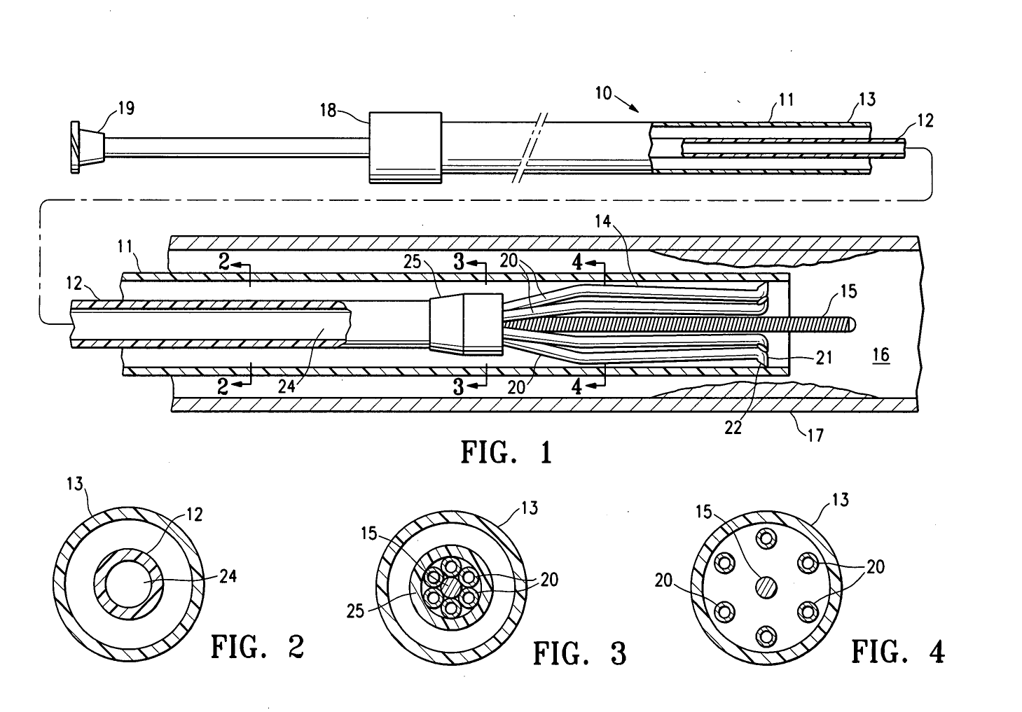

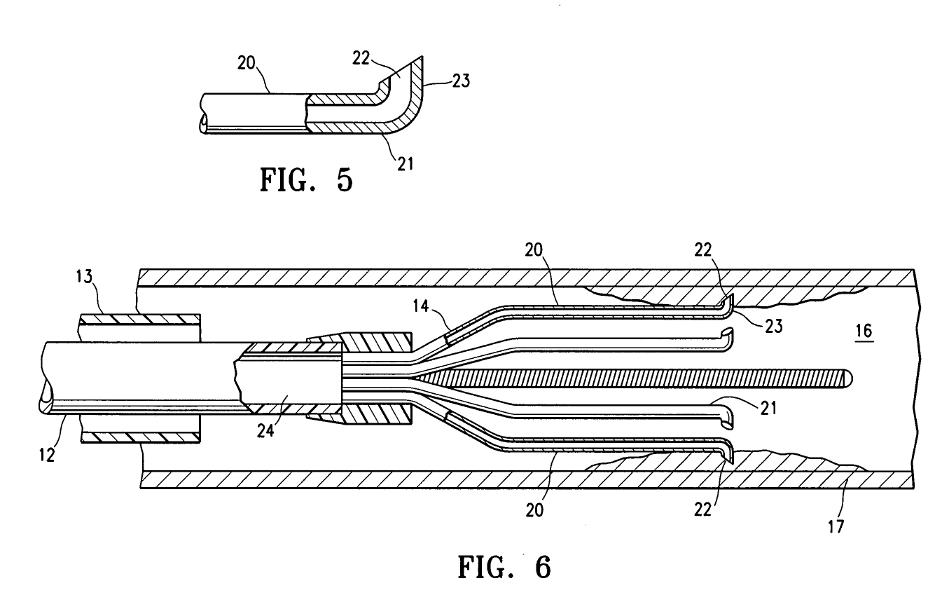

FIG. 1 illustrates an elevational view, partially in section, of a catheter 10 embodying features of the invention, generally comprising an elongated shaft 11 having an inner tubular member 12 and an outer sheath member 13 slidably disposed on the inner member 12, and a self-expanding frame 14 is on a distal shaft section fixedly secured to the shaft inner member 12 and in a radially collapsed configuration slidably disposed in the outer member 13 of the shaft. A floppy tip distal guide member 15 such as a coil is secured at a distal end of the catheter 10 to facilitate maneuvering the catheter 10 within a patient's body lumen. In the illustrated embodiment, the distal guide member coil 15 has a proximal end secured to the distal end of the inner member 12 and a distal end located distal to the frame 14. In an alternative embodiment, the catheter is configured with a guidewire lumen therein for slidably advancing over a conventional guidewire. FIG. 1 illustrates the catheter 10 advanced within a patient's body lumen 16 to a desired location for delivery of an agent to the vessel wall 17.

The frame 14 is formed of a plurality of hollow tubes 20 having joined first ends (which in the illustrated embodiment are the proximal ends of hollow tubes 20) and free second ends 21 (which in the illustrated embodiment are the distal ends of the hollow tubes 20). The free distal end 21 of each hollow tube 20 has an agent delivery port 22 and a hooked distal tip 23, as best illustrated in FIG. 5 showing an enlarged view of a distal end of a single hollow tube 20. At least one agent delivery lumen 24 extends within the shaft. In the embodiment illustrated in FIG. 1, the shaft has a single agent delivery lumen 24, in the inner member 12 of the shaft, in fluid communication with the agent delivery port 22 of each hollow tube 20. In alternative embodiments (not shown), an additional agent delivery lumen(s) is provided in the shaft to allow for the sequential or simultaneous delivery of one or more agents independently through the individual hollow tubes 20 of the frame 14. The frame 14 has a connector 25 fixedly securing the proximal ends of the hollow tubes to the distal end of the inner member 12 of the shaft 11.

The frame 14 radially expands to an expanded configuration by release of a radially restraining force of the shaft outer member 13 (i.e., by slidably displacing the frame 14 and the outer member 13 relative to one another, such that the frame deploys upon becoming distally spaced from the distal end of the outer member). Thus, the frame 14 is biased to automatically radially expand to the expanded configuration when the frame is no longer radially restrained by the outer sheath member 13. The frame is typically deployed to the expanded configuration by proximally withdrawing the outer member 13 while holding the inner member 12 stationary to maintain the position of the frame within the body lumen 16. Although less preferred due to the potential for damage to the vessel wall, the inner member 12 can alternatively or additionally be advanced distally during deployment of the frame 14. A handle 18, similar to conventional handles on self-expanding embolic protection filters and stent delivery systems, is on the proximal end of the catheter 10 for facilitating proximally withdrawing the outer member 13 of the shaft 11 relative to the inner member 12 to deploy the frame 14.

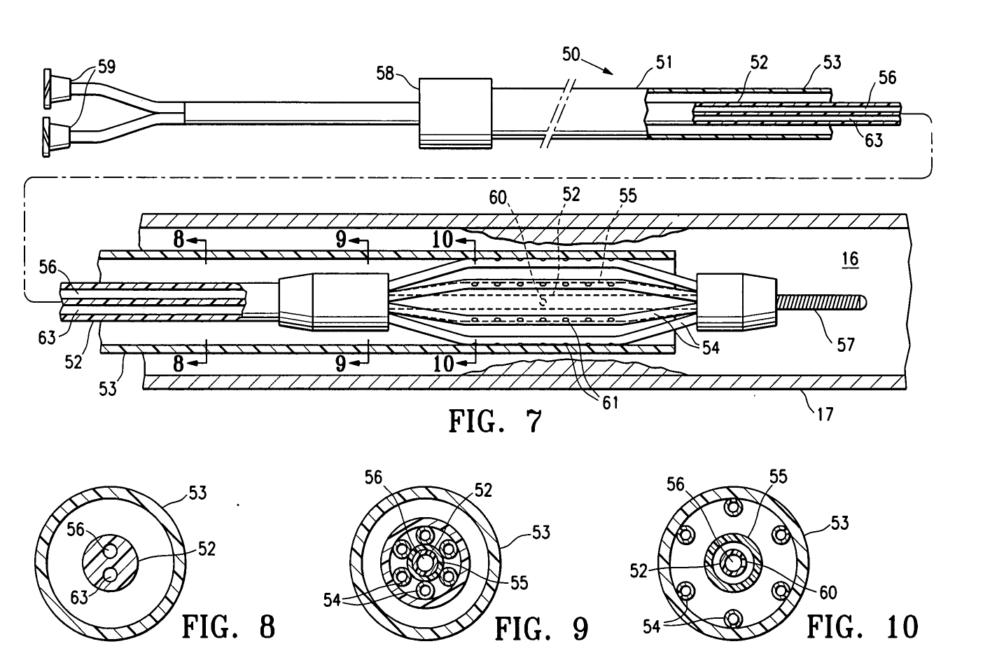

Although not illustrated, in one embodiment, a balloon is provided within the frame 14 which is inflated during deployment of the frame to enhance hook 23 penetration into the vessel wall 17, especially in a very calcified lesion. The balloon would be secured to a distal portion of the shaft 11 such that an interior of the balloon is in fluid communication with an inflation lumen of the shaft 11, similar to the configuration of the embodiment of FIG. 7 discussed below.

The hollow tubes 20 are typically formed of a super-elastic or shape memory alloy or other self-deploying material, such as a nickel-titanium (NiTi) alloy. Additionally, stainless steel or other biocompatible metals or polymers can be utilized to form the hollow tubes 20 of the frame 14. The hooked distal tips 23 are typically formed by bending the distal end of each hollow tube 20, with or without heat, to plastically deform the hollow tube 20 without collapsing the fluid channel therein. As such, the hooked distal tip 23 preferably has a stable shape which does not change upon deployment or subsequent recapture of the frame 14 in the body lumen 16. A sharp end of the hooked distal tip 23 of the hollow tube 20 is typically formed by grinding three-angled facets on each tip.

The frame 14 is preferably designed such that the same size device 10 can perform drug delivery to a variety of different sized vessels, due to the elasticity of the expansion of the frame 14 into contact with the inner surface of the vessel wall.

The hollow tubes 20 are circumferentially spaced and longitudinally extending along the length of the frame 14. In the illustrated embodiment, the frame 14 has a total of six hollow tubes 20. However, more or fewer hollow tubes 20 can be used to form the frame, in order to optimize the drug distribution and targeting of the drug delivery site in the patient's body lumen 16. In one embodiment the hollow tubes 20 have an outer diameter of about 0.13 to about 0.25 mm. The frame 14 is typically configured to radially expand to meet the inner diameter of the specific target vessel, for example at least to an expanded diameter of about 2 to about 5 mm for a coronary artery. In the expanded configuration, the hollow tubes 20 are typically spaced apart by a distance substantially greater than the diameter thereof (depending on the expanded diameter of the frame 14).

FIG. 6 illustrates the operative distal portion of the catheter 10 with the outer member 13 proximally spaced from the frame 14 such that the frame 14 is in the expanded configuration in the vessel 16. The hooked distal tip 23 of each hollow tube 20 is configured for complete or partial penetration of the vessel wall 17 with the frame 14 in the expanded configuration, to imbed the agent delivery port within or outside (through) the vessel wall in the expanded configuration. Thus, as illustrated in FIG. 6, with the agent delivery ports 22 fully imbedded within the vessel wall 17, agent from within the hollow tubes 20 is delivered into the vessel wall through the ports 22. The hooked tips 23 redirect the end of the fluid channel of each hollow tube 20 in a direction radially away from the longitudinal axis of the catheter 10. In the illustrated embodiment, the agent delivery port 22 is similarly oriented radially away from, and not aligned with, the longitudinal axis of the catheter 10, and is formed by a beveled end of the hollow tube 20. The beveled end provides the hollow tube 20 with a large diameter agent delivery port 22 and a penetrating pointed end.

In the expanded configuration, the closed proximal end of the frame 14 remains fixedly secured to the catheter shaft (i.e., the inner member 12), while the open distal end radially expands. The distal end of the frame 14 thus defines an unobstructed fluid path across the distal end of the frame, thereby minimizing any slowing of fluid, e.g., blood, flow within the patient's body lumen 16 due to the presence of the deployed frame 14 therein.

As best illustrated in FIG. 6 showing the frame 14 in the expanded configuration, the frame 14 has a tapered proximal section tapering proximally down to the inner member 12 of the catheter shaft 11, and has a straight central section which extends from the tapered proximal section to the hooked distal tips 23 of the hollow tubes 20. In a preferred embodiment, the straight central section of the frame extends longitudinally at an approximately right angle relative to each hooked tip 23, i.e., the bend in the hollow tube 20 forming the hooked distal tip 23 has an approximately 90 degree angle, although in alternative embodiments it may be greater or less than 90 degrees, e.g., about 45 to about 90 degrees. Although shown, for ease of illustration, in FIG. 6 with a slight gap between the inner surface of the vessel wall 17 and the outer surface of the straight central section of the frame 14, the frame is preferably configured to press against the vessel wall 17 along substantially the entire length of the straight central section of the frame in the expanded configuration.

The length of the straight central section of the frame 14 is typically longer than the length of the proximal tapered section of the frame. In the illustrated embodiment, the hollow tubes 20 all have substantially equal lengths such that the hooked tips 23 are radially aligned at the same location along the length of the frame 14. Thus, the agent is delivered around the circumference of the inner surface of the vessel wall 17 at one transverse location therein in the embodiment of FIG. 6. In alternative embodiments (not shown), the hollow tubes 20 have varied lengths such that the hooked tips 23 are staggered at two or more different locations along the length of the frame 14 for delivering agent circumferentially around and longitudinally along the vessel.

In a presently preferred embodiment, each hollow tube 20 has only one agent delivery port 22. Thus, as illustrated the figures, the hollow tubes 20 are solid-walled tubes from the joined end to the free end thereof, such that the agent delivery port in the free end is the single agent delivery port in each hollow tube 20, although in one embodiment (not shown) each hollow tube is terminated by a plurality of tips each having an agent delivery port 22 therein. Alternatively, the hollow tubes have one or more additional agent delivery ports along the length of the hollow tube proximal to the distal tip port(s), such as an embodiment having smaller diameter ports along the straight length as an option to maximize drug delivery along the vessel length. Each individual hollow tube 20 is typically formed of a single piece of tubing which thus has a unitary structure from the proximal to the distal end of the frame 14, and which has a bent distal end section forming the hooked tip 23, providing superior structural integrity and manufacturability. The transition from the tapered section to the straight section of the frame 14 is formed by a plastically deformed bend in each hollow tube 20, rather than by an articulating joint. The hollow tubes 20 have freedom of movement relative to one another, distal to the end of the shaft inner member 12, which allows the hollow tubes 20 to be circumferentially brought closer together in the frame's collapsed configuration, and to become more circumferentially spaced apart in the frame's radially expanded configuration.

The hollow tubes 20 are secured to the distal end of the inner member 12, typically by adhesive bonding. Thus, adhesive filler (not shown) is typically between and around the outer surface of a distal end section of the tubes 20 to sealingly secure the hollow tubes 20 to the inner member 12, to place the channel within each hollow tube 20 in fluid communication with the agent delivery lumen 24 of the inner member 12. However, a variety of suitable assembly techniques can be used, including crimping and welding to secure the tubes 20 to the shaft inner member 12/connector 25.

A method of delivering an agent to a patient's vessel wall, e.g., an arterial wall of a coronary vessel, using catheter 10 generally comprises advancing the catheter 10 within the patient's vessel to a desired location therein, with the frame 14 radially collapsed within the outer sheath member 13 of the catheter 10 and with the outer sheath member 13 releaseably secured to the inner member 12. At the desired location in the body lumen 16, the frame is slidably displaced relative to the outer member 13 to radially expand the frame 14 into contact with the vessel wall (e.g., by proximally retracting the outer member 13 and/or distally advancing the inner member 12). The frame 14 radially self expands upon release of the radially restraining force of the outer member 13. Upon the self-expansion of the frame 20, the hooked distal tip 23 of one or more of the hollow tubes 20, and preferably of every hollow tube 20, penetrates the vessel wall 17 to thereby imbed the agent delivery port 22 within or through the vessel wall 17. With the agent delivery ports 22 at least in part penetrating the vessel wall 17, agent within the hollow tubes 20 is delivered to the vessel wall through the ports 22. For example, an agent fluid source (not shown), in solution, dispersion, suspension, or other fluid form, including nanoparticles or liposome suspension, is connected to the proximal adapter 19 at the proximal end of the catheter 10, so that the agent is caused to flow through the agent delivery lumen 24 of the shaft and out the agent delivery ports 22 of the frame 14. Similarly, the agent can be preloaded in the distal section of the catheter and pushed or otherwise caused to elute from the frame 14. The agent is thus delivered to the vessel wall 17, which minimizes wash-out of the agent in the vessel lumen 16. The terminology “vessel wall” should be understood to refer to the tissue of the vessel wall, or an implant such as a graft, or various diseased states such as a stenosis or lesion which may be present within the vessel. The agent can be injected into various layers/depths in the vessel wall (e.g., intima, media, adventia, or peri-adventitial space) depending upon the height of the hooked distal tip 23.

FIG. 7 illustrates an alternative embodiment of a catheter 50 embodying features of the invention, generally comprising an elongated shaft 51, a self-expanding frame 54 on a distal shaft section, and a balloon 55 on the distal shaft section and within the frame 54. Non-inflated balloon 55 and a section of the shaft 51, under the frame 54, are illustrated in dashed line in FIG. 7. The shaft generally comprises an inner tubular member 52 having an inflation lumen 56 in fluid communication with the interior of the balloon 55, and an outer sheath member 53 slidably disposed on the inner member 52. Similar to the embodiment of FIG. 1, the self-expanding frame 54 is fixedly secured to the inner member 52, and slidably disposed in the outer sheath member 53 in a radially collapsed configuration which radially expands to an expanded configuration by release of a radially restraining force of the outer sheath member 53. A floppy tip distal guide member 57 such as a conventional guidewire distal tip or coil is secured at a distal end of the catheter to facilitate maneuvering the catheter within a patient's body lumen. In the illustrated embodiment, the distal guide member coil 57 has a proximal end secured to the distal end of the inner member 52 at the distal end of the frame 54. In an alternative embodiment, the catheter is configured with a guidewire lumen therein for slidably advancing over a conventional guidewire. Similar to the embodiment of FIG. 1, at the proximal end of the catheter 50 is a handle 58 on a proximal end of the shaft outer sheath member 53, and proximal adapters 59 in fluid communication with the inner member 52 lumen(s). Handle 58 facilitates slidably displacing the outer sheath member 53 relative to the inner member 52 of the shaft. Proximal adapters 59 are configured for connecting to fluid delivery sources, for agent delivery/balloon inflation.

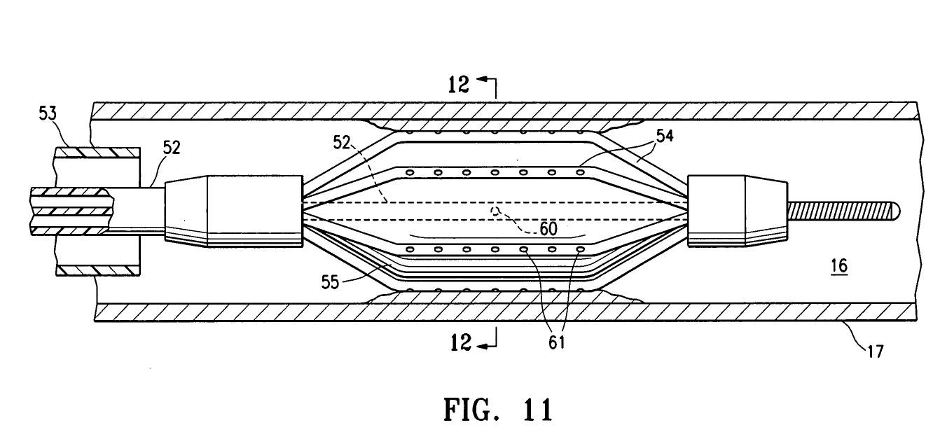

The catheter 50 has a plurality of agent delivery ports 61 along a distal portion of the catheter, configured for delivery of the agent into the patient's blood vessel. FIG. 7 illustrates the catheter 50 with the frame 54 in the collapsed configuration and with the balloon 55 not inflated, and FIG. 11 illustrates the catheter 50 with the frame in the radially expanded configuration and with the balloon inflated. FIGS. 8-10 illustrate transverse cross sections of the catheter 50 of FIG. 7, taken along lines 8-8, 9-9, and 10-10, respectively. Although a gap is shown in FIG. 7 for ease of illustration, the frame 54 typically collapses down to about the outer diameter of the noninflated balloon 55 therein and preferably into contact with the outer surface of the noninflated balloon, to form a low profile configuration for distal advancement within the patient's vessels.

In the embodiment illustrated in FIG. 11, the shaft inner member 52 (illustrated in dashed line in FIG. 11, under the inflated balloon 55), extends through the interior of the balloon 55 to the distal end of the balloon 55 and frame 54. The inner member has an inflation port 60 for delivering inflation fluid from the inflation lumen 56 to the interior of the balloon 55. In the illustrated embodiment, the inflation port 60 is located in a side wall of the inner member 52 at about a half-way point along the length of the balloon 55. However, a variety of suitable shaft configurations can be used including a shaft with an inflation lumen which distally terminates at an inflation port at the proximal end of the balloon. For example, in one embodiment (not shown), the shaft can have an inner tubular member and an outer tubular member (with outer sheath member 53 slidably disposed therearound) with the balloon proximal skirt section sealingly secured to the distal end of the outer tubular member of the shaft and the balloon distal skirt section sealingly secured to the distal end of the inner tubular member of the shaft such that the balloon interior is in fluid communication with an inflation lumen defined by the annular space between the inner and outer tubular members of the shaft.

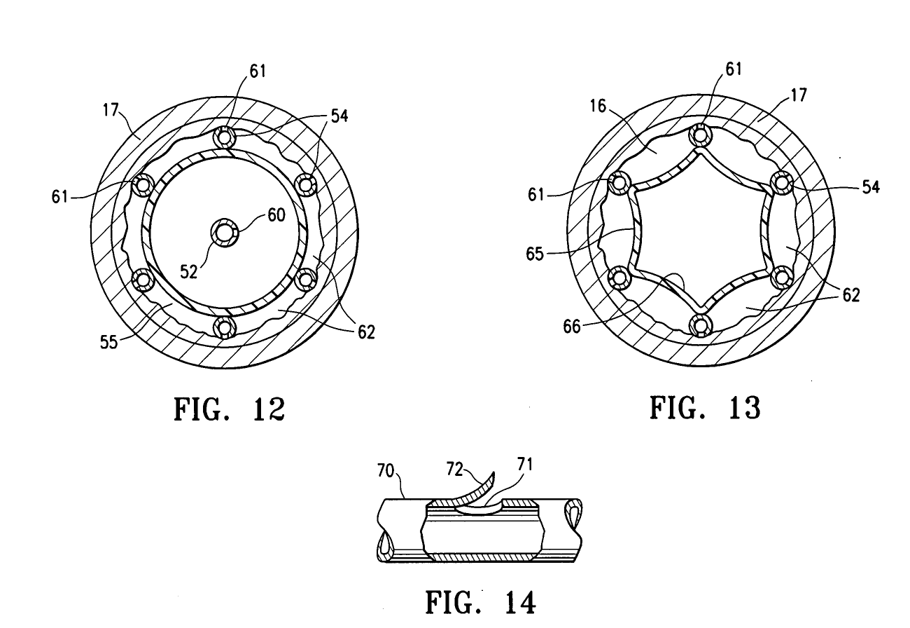

The frame 54 is around the outer surface of the balloon 55 such that when the frame 54 is radially expanded against the patient's vessel wall 17 and the balloon 55 is inflated against an inner surface of the frame 54, the radially expanded frame 54 and balloon 55 together define a plurality of pockets 62 between the vessel wall 17 and the outer surface of the inflated balloon 55. As a result, wash-out of the agent delivered through the ports 61 into the pockets 62 defined by the expanded frame 54 and the inflated balloon 55 is prevented or inhibited. The size and shape of the pockets can vary, depending on the extent to which the balloon expands into the space between adjacent members of the frame (i.e., due to the degree of compliance of the balloon). In the embodiment illustrated in FIG. 12, showing a transverse cross section of the catheter of FIG. 11, the pockets 62 are relatively large, because the balloon 55 has substantially not expanded into the space between adjacent members of the frame 54.

In the illustrated embodiment, the frame 54 has a plurality of agent delivery ports 61 extending along a central (working length) section of the frame 54, with the frame comprising hollow tubes. However, in an alternative embodiment (not shown), the frame is formed by solid wire-like strut members, and the catheter balloon 55 is a porous balloon defining agent delivery ports in the porous wall of the balloon. Thus, it should be understood that the agent delivery ports along the distal portion of the catheter can be in the wall of the balloon and/or in the frame. Regardless of whether the agent delivery ports are in the frame, or the balloon wall, or a combination of both, agent delivered therethrough is preferably retained in the pockets 62 defined by the adjacent surfaces of the vessel wall, expanded frame, and expanded balloon. The catheter 50 thus preferably provides for high drug efficiency/uptake into the vessel wall 17, and low drug wash-out into the systemic circulation. In one embodiment, the agent delivery ports are in the frame 54 only, which allows for agent delivery from the deployed frame for a longer duration while the balloon 55 is deflated to allow blood flow in the vessel 16 if required.

The agent delivery ports 61 on the frame 54 are shown as being visible for ease of illustration and clarity in the views illustrated in FIGS. 7 and 11. However, it should be understood that the ports 61 on the frame 54 are typically on an outer surface of the frame 54 facing/against the inner surface of the vessel wall 17, for delivery of agent directly towards the vessel wall.

In the illustrated embodiment, the inner member 52 has an agent delivery lumen 63 extending adjacent to the inflation lumen 56, in fluid communication with the agent delivery ports 61 of the frame 54. As a result, agent flows from the frame independently of balloon 55 inflation. In the embodiment having a porous balloon, at least an outer-most wall of the balloon 55 is porous to allow for agent to flow from the balloon 55 into the vessel 17. For example, agent infused from the inflation lumen 56 into the balloon interior can be used to inflate the balloon and simultaneously flow across the porous wall of the balloon and into the vessel. However, a variety of suitable drug delivery balloon configurations can be used as are conventionally known including having the balloon inflation be independent of drug infusion by providing a solid-walled inner layer within a porous outer layer of the balloon. Similarly, the operative distal end can be preloaded with the agent by, for example, loading the balloon wall or a reservoir in the balloon with agent which is forced out of the balloon upon inflation thereof, as is conventionally known. The agent delivery lumen 63 of the shaft 51 can optionally be omitted in an embodiment having a preloaded agent delivery operative distal end.

The frame 54 self-expands to the radially expanded configuration, similar to the frame 14 of the embodiment of FIG. 1. Thus, the discussion of the materials and construction of the frame 14 applies as well to the frame 54. The frame 54 expands into contact with the inner surface of the vessel wall 17, with the central (working length) section of the frame 54 impinging against the inner surface of the vessel wall, see e.g., FIG. 11. FIG. 12 illustrates the hollow tubes of frame 54 partially enveloped by the vessel wall 17. The self-expansion of the frame 54 is typically sufficient to radially expand into contact with the inner surface of the vessel wall, and with the hollow tubes/struts of the frame typically pushing into the vessel wall 17, although the balloon 55 can be configured to expand with sufficient radially expansive force to further radially expand a partially expanded frame 54.

Preferably, the frame 54 has about 3 to about 6 hollow tubes/strut members circumferentially spaced around the frame, although a greater or lesser number can be used. In one embodiment the hollow tubes/struts of the frame have an outer diameter of about 0.13 to about 0.25 mm. The frame 54 is typically configured to radially expand at least to the target vessel, for example an expanded diameter of about 2 to about 5 mm for a coronary artery. In the expanded configuration, the hollow tubes/strut members are typically spaced apart by a distance substantially greater than the diameter thereof (depending on the expanded diameter of the frame).

In the illustrated embodiment, the entire length of the balloon expands to the inner diameter of the expanded frame, such that each pocket 62 between any two adjacent hollow tubes/struts of the frame 54 extends the entire expandable length of the frame 54. Alternatively, the balloon 55 can be configured to radially expand to an irregular shape with interspersed portions which do not expand to the inner diameter of the expanded frame 54, such as a lobed-balloon, such that one or more perfusion channels are created along the length of the expanded balloon which allow blood/fluid in the body lumen to flow past the expanded balloon without preventing formation of one or more agent pockets 62. Perfusion can additionally be provided as is conventionally known with perfusion channels (not shown) through the interior of the shaft/balloon which extend between perfusion ports located proximally and distally of the balloon 55. Additionally, the balloon 55 can have a focal/irregularly profiled inflated shape which closes the pockets 65 at the ends of the central working length section of the frame 54. For example, one or both ends of the working length of the balloon 55 can have a radial ridge such as a collar extending around the circumference of the balloon 55, to close the pockets 62 at either end of the length of agent delivery ports 61. Specifically, the profile of the balloon 55 is increased just at the desired location for closing the pockets by adding the collars or otherwise causing the outer surface of the balloon to protrude circumferentially (e.g., with a ring secured within the balloon wall or to an inner or outer surface thereof, or by molding the balloon in a profiled balloon mold, or by other methods of creating a focal/irregularly profiled balloon).

In an alternative embodiment, in place of the balloon 55 as a lining member, the frame 54 is bonded to an outer diameter of a round and soft sleeve material that extends along a length of the frame. The sleeve expands with the frame upon deployment without requiring inflation fluid, and subsequently collapses with the frame after treatment. Moreover, a tubular sleeve conducts blood flow within the lumen defined by the inner surface of the sleeve so that the deployed catheter 50 does not interrupt blood flow through the blood vessel 16. FIG. 13 illustrates a transverse cross section of an alternative embodiment of the catheter 50, having a sleeve 65 secured to the inner surface of the central working length section of the frame 54. The sleeve 65 typically has a length about equal to the length of the central working length section of the frame 54, although it can alternatively have a shorter or longer length. The sleeve can be formed of a variety of suitable polymeric materials, including ePTFE, and can be a porous polymer, or solid-walled (i.e., non-porous). Similar to the embodiment of FIG. 7, one or both ends of the sleeve 65 can have a radial ridge such as a collar extending around the circumference of the sleeve 65, to increase the profile of the sleeve to restrict flow from the ends of the pockets 62. The tubular sleeve 65 which defines an open lumen 66 (i.e., a lumen extending between open proximal and distal ends of the sleeve 65) thus functions as a reservoir collecting extra drug spilled from the ports of the frame 54 and holding it near the vessel wall 17 to enhance drug uptake over longer period of time, while maintaining blood flow within the blood vessel 16 and minimizing drug wash-out.

To increase drug delivery efficiency, the agent delivery ports 61 in the hollow tubes of the frame 54 can be within elevations or at the site of a penetrating hook along the outer hollow tube wall. For example, FIG. 14 illustrates an embodiment in which the frame has a hollow tube 70 with an agent delivery port 71 with a hook 72. In the embodiment of FIG. 14, the hook is formed by laser cutting a triangle tip shape to form a flap from the wall of the hollow tube 70 and lifting up the flap to about a 90 degree angle. Similar to the embodiment of FIG. 1, the hook height controls the depth of hook penetration into the vessel wall.

In one embodiment of a method of the invention, a catheter having a self-expanding frame is slidably displaced in the vessel in the expanded configuration, during an agent delivery procedure, to thereby increase the treated length of the vessel. The catheter useful in the method has a self-expanding frame similar to the frames 14/54 of the embodiments discussed above. For example, frame 54, without or without a lining member, can be deployed by radially expanding the frame to the expanded configuration at a first section of the vessel wall, and agent delivered along the first section of the vessel from the frame's agent delivery ports 61. The deployed frame 54 is then slidably displaced within the vessel in the expanded configuration to a second section, and agent simultaneously or sequentially delivered to the second section of the vessel. The agent delivered to the second section of the vessel can be the same or different than the agent delivered to the first section. In the embodiment having a balloon or sleeve lining member, the pockets formed thereby will contain excess agent within the pockets along the first and second sections of the vessel.

The catheter 10/50 can be used to deliver one or more various agent formulations including liquids, emulsions, nanoparticles, and/or microparticles. Following an agent delivery procedure, the frame 14/54 of catheter 10/50 is collapsed, and the catheter 10/50 withdrawn from the body lumen.

The dimensions of catheter 10/50 are depend upon factors such as the catheter type and the size of the artery or other body lumen through which the catheter must pass. By way of example, the outer sheath member 13 typically has an outer diameter of about 0.025 to about 0.04 inch (0.064 to 0.10 cm), usually about 0.037 inch (0.094 cm), and a wall thickness of about 0.002 to about 0.008 inch (0.0051 to 0.02 cm), typically about 0.003 to 0.005 inch (0.0076 to 0.013 cm). The inner tubular member 12 typically has an inner diameter of about 0.01 to about 0.018 inch (0.025 to 0.046 cm), usually about 0.016 inch (0.04 cm), and a wall thickness of about 0.002 to about 0.004 inch (0.005 to 0.01 cm). The overall length of the catheter 10/50 may range from about 100 to about 150 cm, and is typically about 143 cm. Typically, for coronary arteries, frame 14/54 has a length about 0.8 cm to about 6 cm, and a radially expanded outer diameter of about 2 to about 5 mm.

The shaft tubular members can be formed by conventional techniques, for example by extruding and necking materials already found useful in intravascular catheters such a polyethylene, polyvinyl chloride, polyesters, polyamides, polyimides, polyurethanes, and composite materials. The various components may be joined using conventional bonding methods such as by fusion bonding or use of adhesives.

While the present invention is described herein in terms of certain preferred embodiments, those skilled in the art will recognize that various modifications and improvements may be made to the invention without departing from the scope thereof. For example, the catheters can be designed to have multiple frames (e.g., a bifurcated catheter), and a dilatation/stent delivery balloon can be added to the catheter proximal or distal to the frame to allow the catheter to perform the dual functions of agent delivery and balloon angioplasty/stent delivery. Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

What is claimed is:

1. A catheter configured for delivering an agent to a patient's vessel wall, comprising:
a) an elongated shaft having an inner tubular member with at least one agent delivery lumen, and an outer sheath member slidably disposed on the inner member; and
b) a self-expanding frame on a distal shaft section fixedly secured to the inner member and slidably disposed in the outer member in a radially collapsed configuration which radially expands to an expanded configuration by release of a radially restraining force of the outer member, the frame being formed of a plurality of hollow tubes having joined first ends and free second ends, the free end of each hollow tube having an agent delivery port in fluid communication with the shaft agent delivery lumen and having a hooked tip configured for penetrating the vessel wall in the expanded configuration, to imbed the agent delivery port within the vessel wall or in the periadventitia space outside the vessel wall in the expanded configuration.

2. The catheter of claim 1 wherein the shaft has a single agent delivery lumen in fluid communication with each hollow tube agent delivery port.

3. The catheter of claim 1 wherein the free end of each hollow tube is distal relative to the joined end thereof, such that the frame has a closed proximal end and an open distal end.

4. The catheter of claim 1 wherein the frame in the expanded configuration has a tapered proximal section tapering down to the inner member of the catheter shaft, and has a straight central section which extends from the tapered proximal section to the hooked tips of the hollow tubes and at an approximately 90 degree angle relative to each hooked tip.

5. The catheter of claim 4 wherein the frame is configured to press against the vessel wall along substantially the entire length of the straight central section of the frame in the expanded configuration.

6. The catheter of claim 5 wherein the hollow tubes are solid-walled from the joined end to the free end thereof, such that the agent delivery port in the free end is the single agent delivery port in each hollow tube.

7. The catheter of claim 1 wherein each hollow tube is a single piece of tubing which has a unitary structure from the proximal to the distal end of the frame and which has a bent distal end section forming the hooked tip.

8. The catheter of claim 1 wherein the hollow tubes have substantially equal lengths such that the hooked tips are radially aligned at the same location along the length of the frame.

9. The catheter of claim 1 wherein the hollow tubes have varied lengths such that the hooked tips are staggered at two or more different locations along the length of the frame.

10. The catheter of claim 1 wherein the shaft includes a distal guide member having a proximal end secured to a distal end of the inner member of the shaft, and having a distal end located distal to the frame.

11. The catheter of claim 1 including a balloon on a distal shaft section, fixedly secured to the shaft such that the balloon has an interior in fluid communication with an inflation lumen for inflating the balloon, and the frame is around an outer surface of the balloon.

12. A method of delivering an agent to a patient's vessel wall, comprising:
a) advancing within the patient's vessel a catheter which has an elongated shaft having an inner tubular member with at least one agent delivery lumen and an outer sheath member slidably disposed on the inner member, and a self-expanding frame on a distal shaft section fixedly secured to the inner member in a radially collapsed configuration within the outer member, the frame being formed by plurality of hollow tubes having joined first ends and free second ends, the free end of each hollow tube having an agent delivery port in fluid communication with the shaft agent delivery lumen and having a hooked tip;
b) radially expanding the frame into contact with the vessel wall by slidably displacing the frame relative to the outer member, so that the frame expands to an expanded configuration by release of a radially restraining force of the outer member; and
c) penetrating the vessel wall with the hooked tip of one or more of the hollow tubes, to thereby imbed the agent delivery port within the vessel wall or in the periadventitia space outside the vessel wall; and
d) delivering an agent through the agent delivery ports of the hollow tubes of the frame, to deliver the agent to the patient's vessel wall.

13. The method of claim 12 wherein the frame is positioned around the outer surface of a balloon, and including, after release of the radially restraining force of the outer member, inflating the balloon to push the hooked tips of the frame into the vessel wall.

14. A catheter configured for delivering an agent to a patient's vessel wall, comprising:
a) an elongated shaft having an inner tubular member with an inflation lumen, and an outer sheath member slidably disposed on the inner member;
b) a balloon on a distal shaft section fixedly secured to the inner member such that the balloon has an interior in fluid communication with the inflation lumen for inflating the balloon to an inflated configuration; and
c) a self-expanding frame on the distal shaft section fixedly secured to the inner member, and slidably disposed in the outer member in a radially collapsed configuration which radially expands to an expanded configuration by release of a radially restraining force of the outer member, the frame being around the outer surface of the balloon such that the frame expanded against the vessel wall and the inflated balloon together define a plurality of pockets between the vessel wall and the outer surface of the inflated balloon; and
d) a plurality of agent delivery ports along a distal portion of the catheter, configured for delivery of the agent to the patient's blood vessel, such that wash-out of the agent delivered through the ports into the pockets defined by the expanded frame and the inflated balloon is prevented or inhibited.

15. The catheter of claim 14 wherein the balloon is a porous balloon having a porous wall configured to transport fluid from within the balloon into the patient's vessel, such that at least some of the agent delivery ports are formed by the porous wall of the balloon.

16. The catheter of claim 15 wherein the frame is formed by a plurality of solid strut members.

17. The catheter of claim 15 wherein the shaft has at least one agent delivery lumen, and the frame is formed by plurality of hollow tubes, each hollow tube having a lumen in fluid communication with at least one agent delivery port thereof and with the shaft agent delivery lumen, such that some of the agent delivery ports are in the balloon and some are in the frame.

18. The catheter of claim 17 wherein each hollow tube has a plurality of agent delivery ports spaced along the length of the tube.

19. The catheter of claim 14 wherein the shaft has at least one agent delivery lumen, and the frame is formed by plurality of hollow tubes, each hollow tube having a lumen in fluid communication with at least one agent delivery port thereof and with the shaft agent delivery lumen, such that at least some of the agent delivery ports of the catheter are located in the frame.

20. A catheter configured for delivering an agent to a patient's vessel wall, comprising:
a) an elongated shaft having an inner tubular member with at least one agent delivery lumen, and an outer sheath member slidably disposed on the inner member;
b) a self-expanding frame on the distal shaft section fixedly secured to the inner member, and slidably disposed in the outer member in a radially collapsed configuration which radially expands to an expanded configuration by release of a radially restraining force of the outer member, the frame being formed of a plurality of hollow tubes, each hollow tube having a lumen in fluid communication with the shaft agent delivery lumen, the frame being around and secured to the outer surface of a tubular sleeve such that the frame expanded against the vessel wall and the tubular sleeve together define a plurality of pockets between the vessel wall and the outer surface of the tubular sleeve; and
c) a plurality of agent delivery ports along the frame in fluid communication with the hollow tube lumens of the frame, configured for delivery of the agent to the patient's blood vessel, such that wash-out of the agent delivered through the ports into the pockets defined by the expanded frame and the sleeve is prevented or inhibited.

21. A method of delivering an agent to a patient's vessel wall, comprising:
a) advancing within the patient's vessel a catheter which has an elongated shaft having an inner tubular member and an outer sheath member slidably disposed on the inner member, a self-expanding frame on the distal shaft section fixedly secured to the inner member and around an outer surface of a lining member, the frame being in a radially collapsed configuration within the outer member;
b) radially expanding the frame into contact with the vessel wall by slidably displacing the frame relative to the outer member, so that the frame expands to an expanded configuration by release of a radially restraining force of the outer member, and radially expanding the lining member to an expanded configuration against an inner surface of the expanded frame such that the frame expanded against the vessel wall and the lining member together define a plurality of pockets between the vessel wall and the outer surface of the lining member; and
c) delivering agent through a plurality of agent delivery ports along a distal portion of the catheter, such that wash-out of the agent delivered through the ports into the pockets defined by the expanded frame and the lining member is prevented or inhibited, to deliver the agent to the patient's vessel wall.

22. The method of claim 21 wherein the lining member is an inflatable balloon secured to the shaft, with an inflatable interior in fluid communication with an inflation lumen in the shaft, so that expanding the lining member comprises directing inflation fluid into the balloon interior to inflate the balloon.

23. The method of claim 21 wherein the lining member is a tubular sleeve fixedly secured to the frame, so that the sleeve radially expands and collapses together with the frame.

24. A method of delivering an agent to a patient's vessel wall, comprising:
a) advancing within the patient's vessel a catheter which has an elongated shaft having an inner tubular member and an outer sheath member slidably disposed on the inner member, a balloon on a distal shaft section fixedly secured to the inner member, and a self-expanding frame on the distal shaft section fixedly secured to the inner member and around an outer surface of the balloon, the frame being in a radially collapsed configuration within the outer member;
b) radially expanding the frame into contact with the vessel wall by slidably displacing the frame relative to the outer member, so that the frame expands to an expanded configuration by release of a radially restraining force of the outer member; and
c) inflating the balloon such that the frame expanded against the vessel wall and the inflated balloon together define a plurality of pockets between the vessel wall and the outer surface of the inflated balloon; and
d) delivering agent through a plurality of agent delivery ports along a distal portion of the catheter, such that wash-out of the agent delivered through the ports into the pockets defined by the expanded frame and the inflated balloon is prevented or inhibited, to deliver the agent to the patient's vessel wall.

25. The method of claim 24 wherein the balloon is a porous balloon, and delivering the agent through the agent delivery ports comprises flowing the agent from within the balloon through the porous wall.

26. The method of claim 25 wherein the frame is formed by plurality of hollow tubes, each hollow tube having a lumen in fluid communication with at least one agent delivery port thereof and with an agent delivery lumen in the catheter shaft, such that delivering the agent comprises flowing some of the agent through the balloon agent delivery ports and some of the agent through the frame agent delivery ports.

27. A method of delivering an agent to a patient's vessel wall, comprising:
a) advancing within the patient's vessel a catheter which has an elongated shaft having an inner tubular member and an outer sheath member slidably disposed on the inner member, a self-expanding frame on the distal shaft section fixedly secured to the inner member, the frame being in a radially collapsed configuration within the outer member;
b) radially expanding the frame into contact with a first section of the vessel wall by slidably displacing the frame relative to the outer member, so that the frame expands to an expanded configuration by release of a radially restraining force of the outer member, and delivering agent through a plurality of agent delivery ports along a distal portion of the catheter to deliver the agent to the first section of the vessel wall; and
c) slidably displacing the frame in the expanded configuration longitudinally along the vessel to position the expanded frame at a second section of the vessel wall, and delivering agent through the agent delivery ports to deliver the agent to the second section of the vessel wall.
